(19) United States
(12) Patent Application Publication
(10) Pub. No.: US 2024/0337645 A1
(43) Pub. Date: Oct. 10, 2024

(54) METHODS OF TREATING COMPROMISED LUNG FUNCTION AND ASSESSING CLINICAL IMPROVEMENT

(71) Applicant: The Trustees of Indiana University, Bloomington, IN (US)

(72) Inventors: Nadzeya Marozkina, Indianapolis, IN (US); Benjamin Gaston, Indianapolis, IN (US); Michael Denning Davis, Indianapolis, IN (US)

(21) Appl. No.: 18/294,443

(22) PCT Filed: Aug. 4, 2022
(86) PCT No.: PCT/US22/39430

§ 371 Date: Feb. 1, 2024

(51) Int. Cl. G01N 33/497; A61K 9/00; A61K 33/00; A61K 45/06

(52) U.S. Cl. CPC G01N 33/4975; A61K 9/0078; A61K 33/00; A61K 45/06

(57) ABSTRACT

The present disclosure provides novel approaches novel approaches to the treatment of compromised lung function and methods for measuring or assessing the effectiveness of such treatments. Specifically, the method of assessing the efficacy treatments of compromised lung function where the compromised lung function is caused by primary ciliary dyskinesia and where the methods include measuring fractional nitric oxide concentration and hydrogen peroxide in the exhaled breath of the patient before the initiation of treatment and at various timepoints following the initiation of treatment.

### REFERENCE TO GOVERNMENT GRANTS

This invention was made with government support under HL128192 awarded by National Institutes of Health. The Government has certain rights in the invention.

### FIELD OF THE INVENTION

The general field of the present disclosure are novel approaches to the treatment of compromised lung function and methods for measuring or assessing the effectiveness of such treatments.

### BACKGROUND

Primary ciliary dyskinesia (PCD) is a rare genetic ciliopathy in which mucociliary clearance is disturbed by the abnormal motion of cilia or there is a severe reduction in the generation of multiple motile cilia. Compromised lung function and lung damage ensues due to recurrent airway infections, sometimes even resulting in respiratory failure. So far, no causative treatment is available and treatment efforts are primarily aimed at improving mucociliary clearance and early treatment of bacterial airway infections. Treatment guidelines are largely based on cystic fibrosis (CF) guidelines, as few studies have been performed on PCD.

PCD is thought to be caused by pathologic variants in many different genes that affect the formation, structure, and function of motile cilia. See Leigh et al., “Primary Ciliary Dyskinesia (PCD): A genetic disorder of motile cilia,” (2019) Transl Sci Rare Dis 4: pp. 51-75. PCD may be more common than was previously thought, with a world-wide genetic prevalence estimated to be at least one in 7,600. Hannah et al., “The global prevalence and ethnic heterogeneity of primary ciliary dyskinesia gene variants: a genetic database analysis,” (2022) Lancet Respir Med 10: pp. 459-468. The diagnosis of PCD is challenging, and measurement of nasal NO (nNO) has been widely adopted as a biomarker for disease diagnosis in individuals with features typical of PCD. These features include newborn respiratory distress; chronic nasal and sinus disease; chronic cough, bronchitis, and bronchiectasis; and left-right asymmetry. Though most patients with a confirmed diagnosis of PCD have low nNO, the reasons for this finding are unclear. See Leigh et al. 2019; Hannah et al. 2022; Shapiro et al., “Diagnosis, monitoring, and treatment of primary ciliary dyskinesia: PCD foundation consensus recommendations based on state-of-the-art review,” (2016) Pediatr Pulmonol 51: pp. 115-132; Noone et al., “Primary ciliary dyskinesia: diagnostic and phenotypic features,” (2004) Am J Respir Crit Care Med 169: pp. 459-467; Walker et al., “Upper and lower airway nitric oxide levels in primary ciliary dyskinesia, cystic fibrosis and asthma,” (2013) Respir Med 107: pp. 380-386.

Expression of NO synthase (NOS) isoforms is not consistently different in PCD airways compared to healthy individuals. See Walker et al. 2013. With regard to NOS activity, the inventors have previously reported that decreased ciliary motion prevents airway epithelial endothelial NOS (eNOS) activity, and this difference in activity could contribute to low airway NO value. See Marozkina et al., “Cyclic compression increases F508 Del CFTR expression in ciliated human airway epithelium,” (2019) Am J Physiol-Lung Cell Mol Physiol 317: pp. L247-L258. However, this phenomenon may not fully explain the profoundly low nNO values observed in most PCD patients. An additional potential explanation for low nNO is that NO could be oxidized before leaving the sinuses and nos. See Marozkina et al., “Nitrogen chemistry and lung physiology,” (2015) Annu Rev Physiol 77: pp. 431-452; van der Vliet et al., “Reactive nitrogen species and tyrosine nitration in the respiratory tract: epiphenomena or a pathobiologic mechanism of disease?,” (1999) Am J Respir Crit Care Med 160: pp. 1-9. There is also recent evidence that antigens on the airway epithelial surface increase expression of pro-oxidant enzymes in airway epithelial cells. See Fulcher et al., “Human nasal and tracheo-bronchial respiratory epithelial cell culture,” 2013 Methods Mol Biol 945: pp. 109-121. NO reacts with oxidants, including H₂O₂, O₂, OH· and O₂ itself. Kinetics vary widely and in some cases are not first order (that is, accelerate exponentially at higher reactant concentrations). Products include NO₂, HNO₂⁻/NO₂⁻, NO₃⁻, ONOO⁻/ONOOH and other aqueous nitrogen oxides. See also Gray et al., “Reaction of hydrogen peroxide with nitrogen dioxide and nitric oxide,” (1972) J Phys Chem pp. 1919-1924. All of these oxidative reactions deplete the reactant, NO. Depending on conditions—such as pH—the products can have cytotoxicities: oxidative stress in general, and products of NO oxidation in particular, can injury the airway epithelium. See above and Haddad et al., “Quantitation of nitrotyrosine levels in lung sections of patients and animals with acute lung injury,” (1994) J Clin Invest 94: pp. 2407-2413. One pro-oxidant enzyme is dual oxidase 1 (DUOX1), upregulation of which during antigen stasis is mediated by P2Y and PAR receptors, leading to activation of type 2 alarmins. See van der Vliet et al., “Dual oxidase: a novel therapeutic target in allergic disease,” (2018) Br J Pharmacol 175: pp. 1401-1418; Hristova et al., “Airway epithelial dual oxidase 1 mediates allergen-induced IL-33 secretion and activation of type 2 immune responses,” (2016) J Allergy Clin Immunol 137: pp. 1545-1556.

However, what is needed is an understanding of whether antigen stasis in PCD could damage the PCD epithelium through nitrosative and oxidative stress. This oxidative stress, in turn, could decrease gas phase NO concentrations, increase lung concentrations or oxidants such as hydrogen peroxide and contribute to airway nitrosative stress. Further, what is needed are ways to monitor the effectiveness of treatments that set out to alleviate this airway oxidative stress in patients with compromised lung function. The present invention addresses these needs.

### SUMMARY OF THE INVENTION

The present disclosure provides novel approaches to the treatment of compromised lung function and methods for measuring or assessing the effectiveness of such treatments.

More specifically, current invention provides methods of assessing the efficacy of therapies used in the treatment of a patient with compromised lung function comprising measuring fractional nitric oxide concentration and hydrogen peroxide in the exhaled breath of the patient before the initiation of treatment and at various timepoints following the initiation of treatment. In some embodiments, therapy or therapies comprise antioxidant therapy or the administration of one or more antioxidants.

In any embodiment, an increase in fractional nitric oxide concentration and a decrease in hydrogen peroxide in the exhaled breath of the patient over time indicate positive efficacy of the antioxidant therapy.

In any embodiment wherein one or more antioxidants are administered to a patient with compromised lung function, the one or more of the antioxidants are selected from the group consisting of N-acetylcysteine, Nacystelyn, N-isobutyrlcysteine, carbocisteine, procysteine, erdosteine, thioredoxin, 15d-PGJ2, CDDO-imidazolide, sulforaphane, chalcones, superoxide dismutase, ebselen, molecular hydrogen, celastrol, 2-thioxanthine, glutathione, vitamin A (retinol) β-carotenes, vitamin C (ascorbic acid), vitamin D (cholecalciferol), vitamin E (gamma or alpha tocopherol), lipo-glutathione (glutathione) and coenzyme Q (ubiquinone).

In any of embodiment, the one or more antioxidants are administered by one or more of routes of administration wherein the route of administration is selected from the group consisting of oral, inhalation, intravenous, subcutaneous, sublingual, and transdermal.

In any embodiment disclosed herein, “compromised lung function” is considered to be primary ciliary dyskinesia. In any embodiment the primary ciliary dyskinesia can be exacerbated by one or more of asthma, exposure to cigarette smoke, exposure to atmospheric pollutants, chronic obstructive pulmonary disease, bronchitis, cystic fibrosis, extended post-viral bronchial hyperresponsiveness syndrome, rhinosinusitis, reactive airways dysfunction syndrome, and persistent allergen exposure.

In another embodiment, the invention provides methods of treating compromised lung function in a patient in need thereof wherein the compromised lung function is caused by primary ciliary dyskinesia, the methods comprising a therapy that modulates airway pH. In some embodiments, the therapy that modulates airway pH increases airway pH. In still other embodiments, the therapy that modulates airway pH is nebulized NaHCO₃. In yet other embodiments, the therapy that modulates airway pH is the administration of noninvasive ventilation.

In still other embodiments, the invention provides methods of treating compromised lung function in a patient in need thereof comprising the administration of an agent that modulates airway pH further comprising the administration of one or more of an antioxidant.

In an embodiment of the invention is provided a method of assessing the efficacy of the treatment of compromised lung function in a patient, wherein the compromised lung function is caused by primary ciliary dyskinesia and wherein the treatment comprises administration of a therapy that modulates airway pH. This embodiment of the invention further comprises measuring fractional nitric oxide concentration and hydrogen peroxide in the exhaled breath of the patient before the initiation of treatment and at various timepoints following the initiation of treatment.

In any embodiment, an increase in fractional nitric oxide concentration and a decrease in hydrogen peroxide in the exhaled breath of the patient over time indicate positive efficacy of the of the treatment with an agent that modulates airway pH. In some embodiments, the agent that modulates airway pH increases airway pH. In still other embodiments, the agent is nebulized NaHCO₃. In yet other embodiments, the therapy that modulates airway pH is the administration of noninvasive ventilation.

In any of the methods of the current invention, can further comprise the administration of one or more additional therapeutic agents, including devices and techniques that mechanically mobilize antigens and irritants in the airway, moving them cephalad for expectoration.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIG. 1A-E shows the clearance of fluorescently labeled Derp1 from the apical surface of ciliated HC and PCD human airway epithelial cells (HAECs). FIG. 1A: in this model, HAECs (HC and PCD) were grown on Transwell filters until fully ciliated (4-6) weeks. A fenestration was then cut circumferentially in the filter in a 90° arc, such that the apical (ciliated) surface was in contact with the basolateral medium. Fluorescently labeled Derp1 (Alexa-Fluor 647) was applied to the cell surface, and fluorescent images were obtained from the center of the field obtained at times 0, 1, and 2 hr. Counts of labeled Derp1 were made by three blinded observers. FIG. 1B: negative control (no Derp-1 added) shows essentially no background autofluorescence. FIG. 1C, 1D: representative images of labeled Derp-1 on the surface of HAECs: FIG. 1D=HC's; FIG. 1E=PCD cells (CCNO genotype); FIG. 1E: quantitation of Derp1-f in cells from HC; (green; n=3 filters each from five subjects) and patients with PCD (CCNO; orange, n=9 filters and DNAH11; purple, n=6 filters). In HC cells, label decreased, comparing time 0 to 2 hr (p=0.001). In PCD cells, there was no difference in signal between time 0, time 1 and 2 hr, either for PCD as a whole or for either CCNO or DNAH11 (p=NS). Bars in FIGS. 1B, 1C and 1D=150 um.

FIG. 2A,B shows DUOX1 expression is increased in PCD cells exposed to Derp1. FIG. 2A: immunoblots of DUOX1 expression in HC cells at ALI at baseline and after 24 hr Derp1 exposure (n=3 each independent filters). FIG. 2B: Quantitation of DUOX1 densities. Derp1 increased DUOX expression in HC cells, p=0.02). DUOX1 expression in PCD cells at ALI was elevated at baseline relative to HC (n=3 each; p=0.032). Both at 4 and 24 hr, the increased values of DUOX1 after Derp1 exposure were somewhat higher than those after a similar exposure of HC cells (p=0.044 at 4 hr and p=0.052 at 24 hr).

FIG. 3A,B depicts reduced NO levels in PCD cells. Headspace NO was measured in cells from HC and subjects with PCD at baseline and after 30 min. FIG. 3A: in this model, NO was injected through a septum into sealed vials; initial and final NO concentrations were then measured from the gas over the cells (Headspace). FIG. 3B: headspace final/initial NO concentration ratio after 30 was lower in PCD cells compared to HC airway epithelial cells (n=9 control filters and n=6 PCD filters, three from each subject, p=0.004 for HC vs PCD). Treating PCD cells with SOD increased the final/initial headspace NO ratio (n=3 each, p=0.0019 for PCD+SOD vs. PCD alone). Circles=HC cells; squares=PCD DNAH11; triangles=PCD CCNO.

FIG. 4A-C shows that protein tyrosine nitration is increased in PCD cells. Lysates for cells from HC and PCD subjects were immunoblotted for tyrosine nitrosation without or with treatment with Cytomix. FIG. 4A,B: representative immunoblots of protein 3-nitrotyrosine immunostaining in HC and PCD (CCDC40) cells A. Healthy control cells treated with and without cytomix. FIG. 4B: PCD (CCDC40) cells treated with and without cytomix. M=Molecular weight markers. FIG. 4C: densitometry and quantitation of panel 4A and 4B normalized to β actin (n=3 filters from each cell type; p=0.027 comparing untreated PCD cells to PCD cells treated with cytomix).

FIG. 5A,B shows nasal NO production and breath H₂O₂ production in healthy control and PCD subjects. FIG. 5A: nasal NO production was measured using the standard PCD Foundation protocol by NOA 280i in HC and PCD subjects. Nasal NO production was higher in HC (n=7) than in PCD (n=12; *p<0.0001). FIG. 5B: exhaled breath condensate H₂O₂ production in HC subjects was lower than in PCD subjects (n=5 each, p=0.0027).

FIG. 6 is a model of nitrosative and oxidative stress in PCD

### DETAILED DESCRIPTION

Primary ciliary dyskinesia (PCD) is a rare genetic ciliopathy in which mucociliary clearance is disturbed by the abnormal motion of cilia or there is a severe reduction in the generation of multiple motile cilia. Compromised lung function and lung damage ensues due to recurrent airway infections, sometimes even resulting in respiratory failure. So far, no curative treatment is available and treatment efforts are primarily aimed at improving mucociliary clearance and early treatment of bacterial airway infections. Treatment guidelines are largely based on cystic fibrosis (CF) guidelines, as few studies have been performed on PCD. However, what is needed is an understanding as to how or why PCD airway cells might not efficiently clear antigens from the epithelial surface, resulting in increased oxidative stress. This oxidative stress, in turn, could both decrease gas phase NO concentrations and contribute to airway nitrosative stress. Further, what is needed are ways to monitor the effectiveness of treatments that set out to alleviate this airway oxidative stress in patients with compromised lung function.

The present invention addresses these needs by provides novel approaches to the treatment of compromised lung function and methods for measuring or assessing the effectiveness of such treatments.

More specifically, current invention provides methods of assessing the efficacy of therapies used in the treatment of a patient with compromised lung function comprising measuring fractional nitric oxide concentration and hydrogen peroxide in the exhaled breath of the patient before the initiation of treatment and at various timepoints following the initiation of treatment. In some embodiments, therapy or therapies comprise antioxidant therapy or the administration of one or more antioxidants.

In any embodiment, an increase in fractional nitric oxide concentration and a decrease in hydrogen peroxide in the exhaled breath of the patient over time indicate positive efficacy of the antioxidant therapy.

In any embodiment wherein one or more antioxidants are administered to a patient with compromised lung function, the one or more of the antioxidants are selected from the group consisting of N-acetylcysteine, Nacystelyn, N-isobutyrlcysteine, carbocisteine, procysteine, erdosteine, thioredoxin, 15d-PGJ2, CDDO-imidazolide, sulforaphane, chalcones, superoxide dismutase, ebselen, molecular hydrogen, celastrol, 2-thioxanthine, glutathione, vitamin A (retinol) β-carotenes, vitamin C (ascorbic acid), vitamin D (cholecalciferol), vitamin E (gamma or alpha tocopherol), lipo-glutathione (glutathione) and coenzyme Q (ubiquinone).

In any of embodiment, the one or more antioxidants are administered by one or more of routes of administration wherein the route of administration is selected from the group consisting of oral, inhalation, intravenous, subcutaneous, sublingual, and transdermal.

In any embodiment disclosed herein, “compromised lung function” is considered to be primary ciliary dyskinesia. In any embodiment the primary ciliary dyskinesia can be exacerbated by one or more of asthma, exposure to cigarette smoke, exposure to atmospheric pollutants, chronic obstructive pulmonary disease, bronchitis, cystic fibrosis, extended post-viral bronchial hyperresponsiveness syndrome, rhinosinusitis, reactive airways dysfunction syndrome, and persistent allergen exposure.

In another embodiment, the invention provides methods of treating compromised lung function in a patient in need thereof wherein the compromised lung function is caused by primary ciliary dyskinesia, the methods comprising a therapy that modulates airway pH. In some embodiments, the therapy that modulates airway pH increases airway pH. In still other embodiments, the therapy that modulates airway pH is nebulized NaHCO₃. In yet other embodiments, the therapy that modulates airway pH is the administration of noninvasive ventilation.

In still other embodiments, the invention provides methods of treating compromised lung function in a patient in need thereof comprising the administration of an agent that modulates airway pH further comprising the administration of one or more of an antioxidant.

In an embodiment of the invention is provided a method of assessing the efficacy of the treatment of compromised lung function in a patient, wherein the compromised lung function is caused by primary ciliary dyskinesia and wherein the treatment comprises administration of an a therapy that modulates airway pH. This embodiment of the invention further comprises measuring fractional nitric oxide concentration and hydrogen peroxide in the exhaled breath of the patient before the initiation of treatment and at various timepoints following the initiation of treatment.

In any embodiment, an increase in fractional nitric oxide concentration and a decrease in hydrogen peroxide in the exhaled breath of the patient over time indicate positive efficacy of the of the treatment with an agent that modulates airway pH. In some embodiments, the agent that modulates airway pH increases airway pH. In still other embodiments, the agent is nebulized NaHCO₃. In yet other embodiments, the therapy that modulates airway pH is the administration of noninvasive ventilation.

In any of the methods of the current invention, can further comprise the administration of one or more additional therapeutic agents. including devices and techniques that mechanically mobilize antigens and irritants in the airway, moving them cephalad for expectoration.

### Definitions

Various quantities, such as amounts, sizes, dimensions, proportions, and the like, are presented in a range format throughout this disclosure. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is to describe particular embodiments only and is not intended to be limiting of any embodiment. As used herein, the singular forms “a,” “an”, and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless expressly stated or obvious from context, as used herein, the term “about” in reference to a number or range of numbers is understood to mean the stated number and numbers +/−10% thereof, or 10% below the lower listed limit and 10% above the higher listed limit for the values listed for a range.

In any of the embodiments disclosed herein, the terms “treating” or “to treat” includes restraining, slowing, stopping, or reversing the progression or severity of an existing symptom or disorder.

In any of the embodiments disclosed herein, the term “patient” refers to a human.

### Excipients

Illustrative, non-limiting examples of excipients or carriers include sodium citrate or dicalcium phosphate and/or a) one or more fillers or extenders (a filler or extender may be, but is not limited to, one or more selected from starches, lactose, sucrose, glucose, mannitol, and silicic acid), b) one or more binders (binders may be selected from, but not limited to, carboxymethylcellulose, alginates, gelatin, polyvinylpyrrolidinone, sucrose, and acacia), c) one or more humectants (a humectant may be, but is not limited to, glycerol), d) one or more disintegrating agents (disintegrating agents may be selected from, but are not limited to, agar-agar, calcium carbonate, potato or tapioca starch, alginic acid, silicates, and sodium carbonate), e) one or more solution retarding agents (for example, but not limited to, paraffin), f) one or more absorption accelerators (selected from, but not limited to, quaternary ammonium compounds), g) one or more wetting agents (for example, but not limited to, acetyl alcohol and glycerol monostearate), h) one or more absorbents (selected from, but not limited to, kaolin and bentonite clay), and i) one or more lubricants (selected from, but not limited to, talc, calcium stearate, magnesium stearate, solid polyethylene glycols, and sodium lauryl sulfate). In the case of capsules, tablets and pills, for example, the dosage form may also comprise buffering agents.

### “Effective or Therapeutic Amount”

Effective or therapeutic amounts of any of the drugs or pharmaceutical compositions of this disclosure include any amount sufficient to inhibit (e.g., slow or stop) the progression of a neurodegenerative disorder. In some embodiments, effective amounts of the compositions include any amount sufficient to inhibit (e.g., slow or stop) the deterioration of the muscular function of a patient.

The amount of the active ingredient that may be combined with the optional carrier materials to produce a single dosage form may vary depending upon the host treated and the particular mode of administration. The specific dose level for any particular patient may depend upon a variety of factors, including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

### Pharmaceutically Acceptable Carrier

As used herein, the term “pharmaceutically acceptable carrier” means a non-toxic, inert solid, semi-solid or liquid filler, diluent, encapsulating material or formulation auxiliary of any type. In the treatment methods contemplated by the present disclosure, the disclosed agents may be used alone or in compositions together with a pharmaceutically acceptable carrier or excipient, such as saline. For example, an oral dosage form composition may comprise one or more of the disclosed agents in addition to a pharmaceutically acceptable carrier.

An inhalation dosage form composition may one or more of the disclosed agents in addition to a pharmaceutically acceptable carrier. A composition for buccal administration may comprise one or more of the disclosed agents in addition to a pharmaceutically acceptable carrier. A composition for nasal administration may comprise one or more of the disclosed agents in addition to a pharmaceutically acceptable carrier. Further, if a transdermal patch is used as the method of administering one or more of the disclosed agents to the patient, the transdermal patch may comprise one or more of the disclosed agents in addition to a pharmaceutical acceptable carrier.

Some examples of materials which can serve as pharmaceutically acceptable carriers are sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil; safflower oil; sesame oil; olive oil; corn oil and soybean oil; glycols, such as propylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol, and phosphate buffer solutions, as well as other non-toxic compatible lubricants, such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, the contents of which are expressly incorporated herein by reference.

### Oral Dosage Forms

In certain embodiments, one or more of the disclosed agents may be orally administered to be ingested by humans and other animals. Solid dosage forms for oral administration include, as illustrative but non-limiting examples, capsules, tablets, pills, powders, thin films and granules. In solid dosage forms, the active compound may be mixed with at least one inert, pharmaceutically acceptable excipient or carrier, as described in more detail below. As illustrative, non-limiting examples, an oral dosage form of the presently disclosed pharmaceutical composition may be mixed with about 0.1% to about 1%, such as about 0.5%, methyl cellulose.

### Stabilizers

A composition, formulation, or dosage form herein may further comprise one or more of the disclosed agents and one or more stabilizers. As used herein, a stabilizer is a substance that extends the time before which one or more of the disclosed agents in a composition is converted to a salt in the environment in which the formulation or dosage form is administered, in comparison to the conversion in its absence. Non-limiting examples of stabilizers include phosphatidyl choline, phosphatidyl inositol, phosphatidyl ethanolamine, or other phospholipids. A composition, formulation, or dosage form further comprising one or more stabilizers may be administered in any one of the methods herein. A stabilizer may be present in an amount of about 50 mg to about 1000 mg in a composition, formulation, or dosage form herein. In some embodiments, the stabilizer may be present in an amount ranging from about 50 mg to about 500 mg or about 50 mg to about 100 mg.

### Liquid Dosage Forms

Liquid dosage forms for oral administration include, but are not limited to, pharmaceutically acceptable emulsions, microemulsions, solutions, suspensions, syrups and elixirs. In addition to the active compounds, the liquid dosage forms may comprise one or more inert diluents. The inert diluents may be selected from those commonly used in the art. Illustrative, non-limiting examples of inert diluents include water or other solvents, solubilizing agents and emulsifiers including, but not limited to, ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The oral compositions may comprise one or more adjuvants. Illustrative, non-limiting examples of adjuvants include wetting agents, emulsifying and suspending agents, sweetening, flavoring, and perfuming agents.

### Nebulization and Inhalation:

In some embodiments, the pharmaceutical composition may be administered to a patient as nasal drop (intranasally) or using a nebulization technique. A nebulizer may be used to change a liquid solution of a pharmaceutical composition into a fine mist that may be inhaled by a patient. The inventor determined numerous benefits of these techniques. For example, the dosage of the pharmaceutical composition can be significantly decreased when either nasal drop or nebulization is used as the delivery method. In some instances, the dosage may be reduced by about one tenth or one twentieth as compared to, for example, injections, oral administration/ingestion of a liquid solution or oral administration/ingestion of a pill. Moreover, using a nebulization technique or nasal drop bypasses the digestive system whereas ingesting a pill or liquid solution of a pharmaceutical composition sends the composition to the digestive system. Finally, using either a nasal drop or nebulization technique allows the pharmaceutical composition to travel from the olfactory bulb directly to the brain.

In some embodiments, the nebulized pharmaceutical composition may be inhaled through one or both of the mouth or the nasal passage. Without being bound to any theory, it is believed that nasal administration of the composition can take advantage of “nose-to-brain” (N2B) transport systems in which several possibilities exist for bypassing the blood-brain-barrier for direct delivery to the brain. These include the draining of drugs absorbed in the nasal mucosa into the sinus and eventually to the carotid artery, where a “counter-current transfer” from venous blood to the brain may occur. Lymphatic drainage into the perivascular space from the olfactory trigeminal nerves between the central nervous system (CNS) have also been postulated as the mechanism of N2B transport. Additionally, a buffer used to increase airway pH could be used as a diluent for an inhaled antioxidant compound.

Nebulizers are known in the art and the invention of the present disclosure can be used in connection with any nebulizer. For example, the pharmaceutical composition disclosed herein may be nebulized with an inhaler or a Buxco® Inhalation Tower All-In-One Controller.

### Modulation of Airway pH

Embodiments of the current invention include the administration of a therapy that modulates airway pH. The inventors contemplate that therapy that modulates airway pH can include administration of an agent such as nebulized or aerosolized NaHCO₃. In still other embodiments the therapy that modulates airway pH can be the use of noninvasive ventilation. It is understood that noninvasive ventilation includes any kind of mechanical support know in the art that provides noninvasive positive-pressure ventilation. Noninvasive positive-pressure ventilation includes various techniques for augmenting alveolar ventilation without an endotracheal airway. The clinical application of noninvasive ventilation by use of continuous positive airway pressure alone is referred to as “mask CPAP,” and noninvasive ventilation by use of intermittent positive-pressure ventilation with or without continuous positive airway pressure is called noninvasive positive-pressure ventilation. See Penuelas et al., “Noninvasive positive-pressure ventilation in acute respiratory failure,” (2007) CMAJ 177: pp. 1211-1218.

### General Methods and Assays

### Human Studies

Studies involving human subjects were reviewed and approved by the relevant institutional review boards (IRBs).

### Primary Cell Culture

Primary normal human airway epithelial cells (NHAECs) were grown at air-liquid interface (ALI) on Transwell filters (Corning, 3460, Corning NY) from nasal brushings and from explanted lung tissue as previously describe. See Marozkina et al., 2019; van der Vliet et al. 1999. Nasal brushes were obtained from non-smoking healthy controls (HCs) and from PCD subjects in our PCD clinics, and normal bronchial cells were purchased commercially (Lifeline Cell Technologies, Frederick, MD). Subjects with PCD carried variants in two disease-causing alleles in trans for CCNO (consanguineous: homozygous for C807T>A); CCDC40 (248delC and 3097A>T); and DNAH11 4453C>T and 13331G>C). Cells were grown at ALI until fully differentiated as pseudostratified epithelial cells (˜6 weeks) as previously described. See above. The specific PCD genotypes were chosen both because they represented heterogeneous pathogenic mechanisms, and because patients with these genotypes were available for participation. All cells except those from the CCNO patient expressed abundant cilia. See Hristova et al. 2016. In the studies of tyrosine nitration, we compared cells with and without iNOS stimulation using cytomix (10 ng/ml each: tumor necrosis factor-α, TNFα; interleukin-1β, IL1β; interferon-γ, IFN-γ) for 5 hr uniformly to optimize NOS activity. See Asano et al., “Constitutive and inducible nitric oxide synthase gene expression, regulation, and activity in human lung epithelial cells.” (1994) Proc Natl Acad Sci USA 91: pp. 10089-10093; De Sanctis et al., “Contribution of nitric oxide synthases 1, 2, and 3 to airway hyperresponsiveness and inflammation in a murine model of asthma,” (1999) J Exp Med 189: pp. 1621-1630. In some experiments, cells were incubated with or without 3,000 units of Cu/Zn superoxide dismutase (SOD). See Johnson et al., “Accelerated s-nitrosothiol breakdown by amyotrophic lateral sclerosis mutant copper, zinc-superoxide dismutase,” (2001) J Biol Chem 276: pp. 39872-39878.

### Antigen Clearance Assay

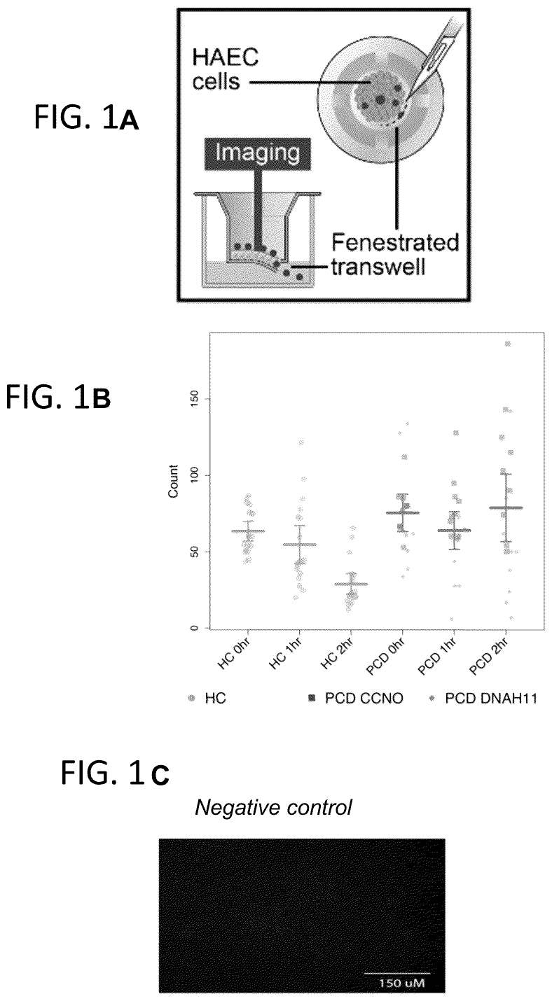

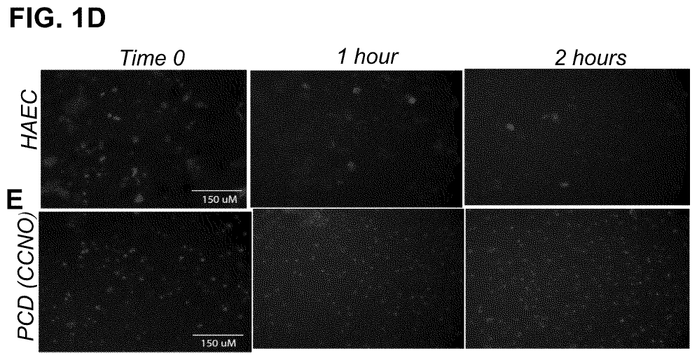

Dermatophagoides pteronyssinus allergen 1 (Derp1) was purchased from Indoor Biotechnologies (Charlottesville, VA; RP-DP1D-1) and conjugated to Alexa Fluor 647 using the manufacturer's protocol (ThermoFisher Scientific; A30009). NHAECs and PCD cells were grown on filters for 6 weeks. In some experiments, filters were fenestrated circumferentially using a scalpel to produce a continuous opening along the edge of the membrane, (along 90° C. arc; see FIG. 1A, allowing a lip of membrane to hang into the medium, and permitting direct communication between the ciliated cells on top of the filter and the basolateral medium. Cells were then exposed apically to fluorescently labeled Dep1 (Derp1-f), which was imaged in the center (unfenestrated part) of the filter using an EVOS M5000 microscope at time 0, 1 and 2 h. Fluorescent labels from photomicrographs were counted by three blinded observers.

### Immunoblots

3-Nitrotyrosine: Proteins from cell lysates (50 ug total) were separated on a 4-15% TGX gel (BioRad, 4561084) and transferred to a PVDF membrane (Biorad, 1704156). To detect 3-Nitrotyrosine, whole cell lysates from NHAECs and CCDC40 cells were immunoblotted for 3-nitrotyrosine, a stable protein modification caused by tyrosine nitration, using rabbit anti-nitrotyrosine antibody (Cell Signaling, #9691, 1:1000 in BSA). Levels were normalized to 3-actin using rabbit anti β-actin antibody (Cell Signaling, #4970, 1:2000 in milk). Secondary antibodies were purchased from Santa Cruz (Goat anti-rabbit, #sc2357, 1:3000). Blots were visualized on the Bio Rad Chemidoc system.

DUOX1: Expression was measured using the ProteinSimple Jess capillary electrophoreses system (San Jose; CA). 1.5 ug of cell lysate was separated according to the manufacturer's protocol, and DUOX1 was detected using a primary goat anti DUOX1 antibody (Origene, #TA320203, 1:50), and anti-goat secondary antibody (ProteinSimple Cat #DM-006). DUOX1 levels were normalized to total protein (ProteinSimple Cat #DM-TP01).

Nitrate (NO₃⁻) and Nitrite (NO₂⁻) Assays

NO₃⁻ was measured colorimetrically after reduction by vanadium chloride as previously described. See for example: Marozkina et al. 2015. NO₂⁻ was measured colorimetrically, but without reduction.

### Headspace NO Assay

Cells from HC or from subjects with PCD (DNAH11, CCNO) were placed in media in an 8 mL glass vial with a sealed membrane cap containing 1 mL of medium, one membrane per vial, with or without 3000 units of SOD. One mL of 40 ppm NO was injected into the sealed gas compartment above the cells and medium (the headspace) and the sample gently vortexed (5 sec). Samples for gas analysis were withdrawn at time 0 and at 30 min. NO was measured by a Sievers NOA 280i analyzer (Zysense, Waddington, NC). The first peak was measured (closing the stopcock at the end of the injection generated a second, artifactual, peak). Comparison of peaks between time 0 (t₀) and time (t_{30 min}) was done.

### Nasal nNO Assay

Nasal nNO was measured as described previously (3) in seven HC volunteers and 12 subjects who met clinical criteria for PCD using a Sievers NOA 280i. See Shapiro et al. 2016.

### Exhaled Breath Condensate (EBC) Collection and Analysis

H₂O₂ levels in EBC were measured from five healthy HCs and from five subjects who met clinical criteria for PCD using the Inflammacheck® point-of-care device (Exhalation Technologies, Cambridge, UK) according to manufacturer's recommendations. Collection was performed in accordance with the European Respiratory Society/American Thoracic Society technical standards. See Horvith et al., “A European Respiratory Society technical standard: exhaled biomarkers in lung disease,” (2017) Eur. Resp. Jour 49: p. 1600965.

### Statistical Analysis

For data with a Gaussian distribution, we used the Student's unpaired, two-tailed t-test. For non-Gaussian data sets, as determined by the Shapiro-Wilk test, we used the Wilcoxon rank-sum test. For repeated measures, we used a generalized estimating equation (GEE) to conduct robust comparison between groups and measurements. Multiple testing correction was performed following the Tukey's procedure when considering pair-wise comparisons and the Bonferroni correction for the rest.

Further reference is made to the following experimental examples.

### EXAMPLES

The following examples are provided for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

Studies on Antigen Stasis and Airway Nitrosative Stress in Human Primary Ciliary Dyskinesia

### Antigen Stasis on PCD Epithelial Cells

To assess antigen stasis on the PCD epithelium, the inventors further studied loss of Derp-f from the surface of airway epithelial cells from HC and PCD subjects cultured at ALI. Two sets of PCD disease-causing variants were used for these studies: i) DNAH11, which results in cilia with rapid, ineffective beating, and ii) CCNO, which results in reduced generation of motile cilia. See Wallmeier et al., “Mutations in CCNO result in congenital mucociliary clearance disorder with reduced generation of multiple motile cilia,” (2014) Nat Genet 46: pp. 646-651; Schultz et al., “Two novel mutations in the DNAH11 gene in primary ciliary dyskinesia (CILD7) with considerable variety in the clinical and beating cilia phenotype,” (2020) BMC Med Genet 21: pp. 237-237. The Transwell filters were fenestrated to allow the movement of Derp1-f from the surface to the basal compartment as shown in FIG. 1A-E.

FIG. 1A-E shows the clearance of fluorescently labeled Derp1 from the apical surface of ciliated HC and PCD human airway epithelial cells (HAECs). FIG. 1A: in this model, HAECs (HC and PCD) were grown on Transwell filters until fully ciliated (4-6) weeks. A fenestration was then cut circumferentially in the filter in a 90° arc, such that the apical (ciliated) surface was in contact with the basolateral medium. Fluorescently labeled Derp1 (Alexa-Fluor 647) was applied to the cell surface, and fluorescent images were obtained from the center of the field obtained at times 0, 1, and 2 hr. Counts of labeled Derp1 were made by three blinded observers. FIG. 1B: negative control (no Derp-1 added) shows essentially no background autofluorescence. FIG. 1C, 7D: representative images of labeled Derp-1 on the surface of HAECs: FIG. 1D=HC's; FIG. 1E=PCD cells (CCNO genotype); FIG. 1E: quantitation of Derp1-f in cells from HC; (green; n=3 filters each from five subjects) and patients with PCD (CCNO; orange, n=9 filters and DNAH11; purple, n=6 filters). In HC cells, label decreased, comparing time 0 to 2 hr (p=0.001). In PCD cells, there was no difference in signal between time 0, time 1 and 2 hr, either for PCD as a whole or for either CCNO or DNAH11 (p=NS). Bars in FIGS. 1B, 1C and 1D=150 um.

In cell preparations from HC, a mean of 59%±7% of Derp1 was cleared from the apical surface of the epithelium after 2 hr. Derp1-f was poorly cleared from filters with either DNAH11 or CCNO genotype. The antigen clearance was 27%±26%. The clearance at 2 hr in HC cells was significantly greater than from PCD cells. The study was not extended beyond 2 hr as the baseline fluorescent signal became attenuated in all cells after 2 hr.

Upregulation of DUOX1 at Baseline and with Derp1 Stasis in PCD Airway Epithelial Cells

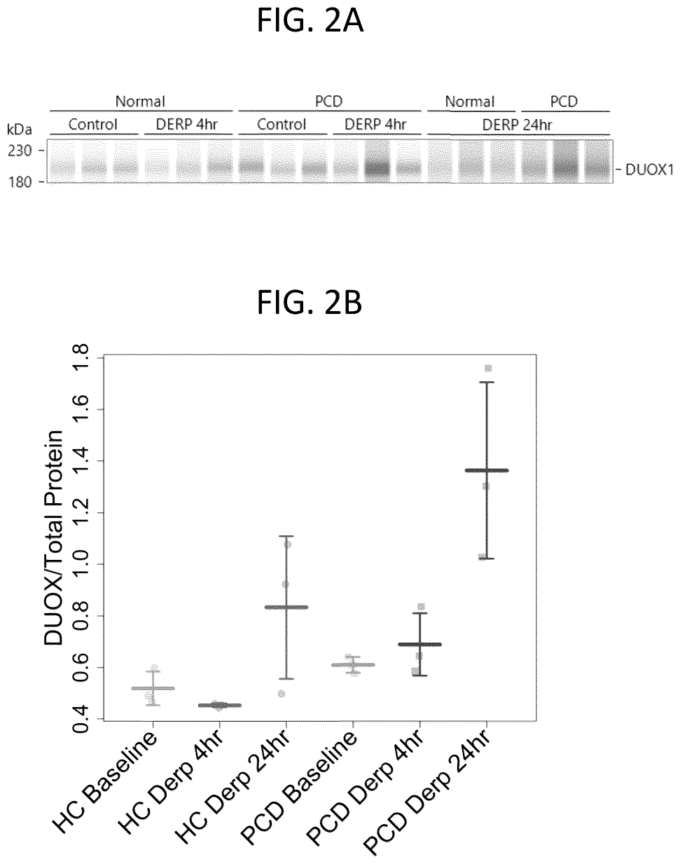

Derp1 increased DUOX1 expression relative to β actin in HC cells at ALI after 24 hr exposure, ranging from 0.45±0.004 to 0.83±0.14 as shown in FIG. 2A,B.

FIG. 2A,B shows DUOX1 expression is increased in PCD cells exposed to Derp1. FIG. 2A: immunoblots of DUOX1 expression in HC cells at ALI at baseline and after 24 hr Derp1 exposure (n=3 each independent filters). FIG. 2B: Quantitation of DUOX1 densities. Derp1 increased DUOX expression in HC cells, p=0.02). DUOX1 expression in PCD cells at ALI was elevated at baseline relative to HC (n=3 each; p=0.032). Both at 4 and 24 hr, the increased values of DUOX1 after Derp1 exposure were somewhat higher than those after a similar exposure of HC cells (p=0.044 at 4 hr and p=0.052 at 24 hr).

This is consistent with previous data. See van der Vliet et al., “Dual oxidase: a novel therapeutic target in allergic disease,” (2018) Br J Pharmacol 175: pp. 1401-1418; Hristova et al., “Airway epithelial dual oxidase 1 mediates allergen-induced IL-33 secretion and activation of type 2 immune responses,” (2016) J Allergy Clin Immunol 137: pp. 1545-1556.

DUOX1/β actin expression in PCD cells (0.61±0.016) was elevated at baseline relative to that in HC cells. Compared to time 0 hr, at 4 hr and 24 hr, DUOX1/β actin increased in PCD cells (from 0.61±0.016 to 0.69±0.062 at 4 hr and 1.36±0.17 at 24 hr). These levels in PCD cells were higher that the increases in DUOX1/β actin in HC cells at 4 hr (0.45±0.004, p=0.044); and there was a trend toward a difference at 24 hr (0.83±0.14, p=0.052). Taken together, these data suggest that elevated DUOX1 expression in the airway epithelium of PCD patients may, in part, be the result of an inability to efficiently clear antigens from its surface.

### Loss of NO in PCD Cells

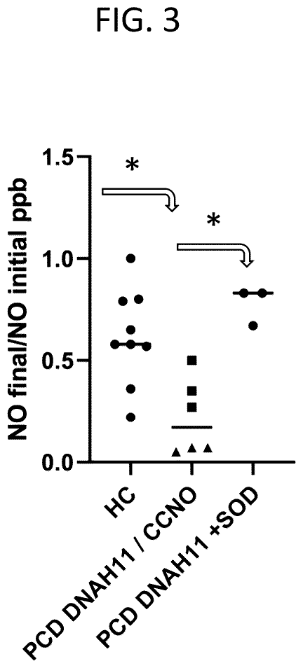

Loss of NO in the epithelium was assessed as the ratio of final to initial headspace NO collected in sealed glass chambers containing filters of mature ALI cultures and medium (see FIG. 3A,B).

FIG. 3A,B depicts reduced NO levels in PCD cells. Headspace NO was measured in cells from HC and subjects with PCD at baseline and after 30 min. FIG. 3A: in this model, NO was injected through a septum into sealed vials; initial and final NO concentrations were then measured from the gas over the cells (Headspace). FIG. 3B: headspace final/initial NO concentration ratio after 30 was lower in PCD cells compared to HC airway epithelial cells (n=9 control filters and n=6 PCD filters, three from each subject, p=0.004 for HC vs PCD). Treating PCD cells with SOD increased the final/initial headspace NO ratio (n=3 each, p=0.0019 for PCD+SOD vs. PCD alone). Circles=HC cells; squares=PCD DNAH11; triangles=PCD CCNO.

The ratio of final/initial headspace NO lost over PCD cells (0.22+0.19) was lower than that over HC airway cells (0.62±0.23). Compared to no treatment, treating PCD cells with antioxidant SOD increased headspace NO final to initial ratio to 0.78±0.092. (FIG. 3B).

### NO Oxidation Products in PCD Cells

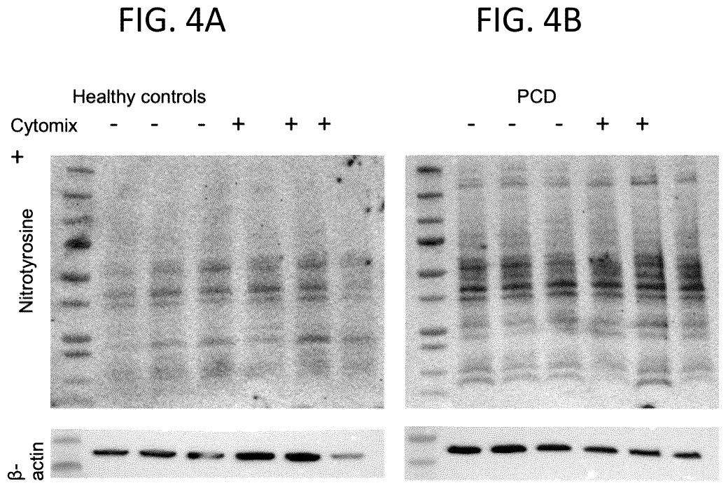

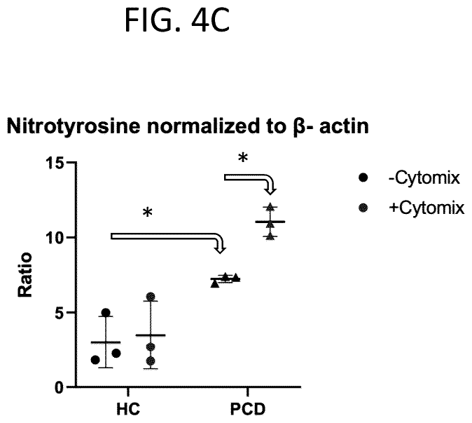

The inventors further studied the cellular “footprint” of NO oxidation, protein 3-Nitrotyrosine (3NT) immunostaining, in HC and PCD (CCDC40) cells at ALI, with and without iNOS upregulation. See van der Vliet et al. 2018; Asano et al., “Constitutive and inducible nitric oxide synthase gene expression, regulation, and activity in human lung epithelial cells,” (1994) Proc Natl Acad Sci USA 91: pp. 10089-10093. The results are shown in FIG. 4A-C.

FIG. 4A-C shows that protein tyrosine nitration is increased in PCD cells. Lysates for cells from HC and PCD subjects were immunoblotted for tyrosine nitrosation without or with treatment with Cytomix. FIG. 4A,B: representative immunoblots of protein 3-nitrotyrosine immunostaining in HC and PCD (CCDC40) cells. Healthy control cells treated with and without cytomix. FIG. 4B: PCD (CCDC40) cells treated with and without cytomix. M=Molecular weight markers. FIG. 4C: densitometry and quantitation of panel 10A and 10B normalized to β actin (n=3 filters from each cell type; p=0.027 comparing untreated PCD cells to PCD cells treated with cytomix).

At baseline, HC immunostaining for 3NT was less in HC cells than in PCD cells (FIG. 4A,B). After 5 hr of cytomix exposure, NO oxidation was increased in PCD compared to HC cells. Additionally, concentrations of the NO oxidation product NO₃⁻ tended to be lower in HC epithelial cells than in PCD (CCDC40) cells (0.038±0.024 vs 0.032.±0.05 μM/μg protein), though this difference was not statistically significant. SOD, on the other hand, completely eliminated NO₃⁻ from PCD cells (decrease to 0 μM/μg protein). Nitrite was less in HC than in PCD (CCDC40) cells (0.018±0.002 vs 0.034±0.004 μM/μg). NO₂⁻ was not affected by SOD, likely because the principal effect of SOD is to prevent ONOO⁻/NO₃⁻ formation, not NO oxidation to NO₂⁻ by oxygen. Taken together, these findings suggest that NO is oxidized in the PCD epithelium relative to control epithelium.

### In Vivo Measurements of Airway Oxidation and of Gas Phase NO

In contrast to evidence for increased aqueous phase concentrations of NO oxidation products in PCD cells described above, gas phase concentrations were quite low in PCD relative to HC.

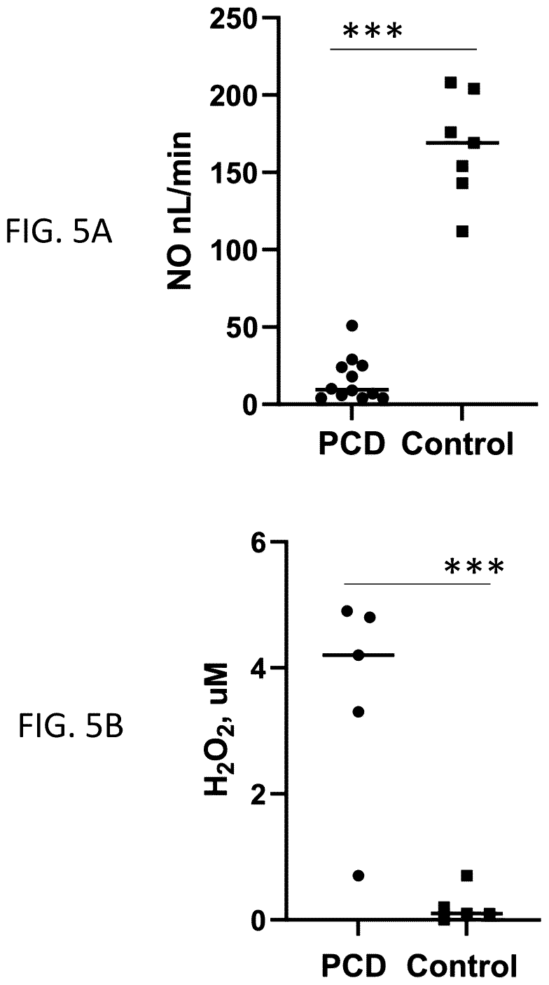

FIG. 5A,B shows nasal NO production and breath H₂O₂ production in healthy control and PCD subjects. FIG. 5A: nasal NO production was measured using the standard PCD Foundation protocol by NOA 280i in HC and PCD subjects. Nasal NO production was higher in HC (n=7) than in PCD (n=12; *p<0.0001). FIG. 5B: exhaled breath condensate H₂O₂ production in HC subjects was lower than in PCD subjects (n=5 each, p=0.0027).

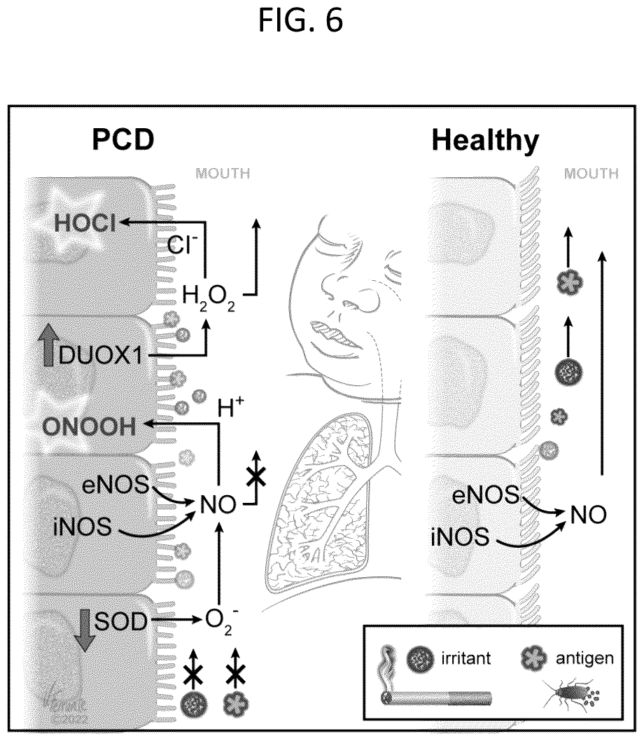

It was found that nNO was much higher in HC subjects (167+/−33 nL/min) than in PCD subjects (15.9+/−14 nL/min) (FIG. 5A). This also confirmed previous observations. See Shapiro et al., “Diagnosis, monitoring, and treatment of primary ciliary dyskinesia: PCD foundation consensus recommendations based on state of the art review,” (2016) Pediatr Pulmonol 51: pp. 115-132; Noone et al., “Primary ciliary dyskinesia: diagnostic and phenotypic features,” (2004) Am J Respir Crit Care Med 169: pp. 459-467. Because DUOX1 generates H₂O₂, the inventors next measured EBC for H₂O₂ in vivo. This was lower in HC than in PCD subjects (0.22+/−0.5 uM vs. 3.58+/−2.5 uM) (FIG. 5B). As discussed in more detail below, low NO (g) levels in the setting of high concentrations of cellular/aqueous NO oxidation products is consistent with loss of airway NO to oxidation in PCD. See FIG. 6.

### SUMMARY

Proteins encoded by disease causing variants in many different genes are associated with PCD. See Leigh et al., “Primary Ciliary Dyskinesia (PCD): A genetic disorder of motile cilia,” (2019) Transl Sci Rare Dis 4: pp. 51-75; Wallmeier et al., “Mutations in CCNO result in congenital mucociliary clearance disorder with reduced generation of multiple motile cilia,” (2014) Nat Genet 46: pp. 646-651; Davis et al., “Primary Ciliary Dyskinesia: Longitudinal Study of Lung Disease by Ultrastructure Defect and Genotype,” (2019) Am J Respir Crit Care Med 199: pp. 190-198; Hannah et al., “The global prevalence and ethnic heterogeneity of primary ciliary dyskinesia gene variants: a genetic database analysis,” (2021) Lancet Respiratory Medicine (2021); Schultz et al. 2020. Mutations in these genes cause several different defects in proteins—including defective synthesis, trafficking, formation, repair, structure and coordinated function of cilia-related proteins. These all result in insufficient ciliary function, the defining feature of PCD. Most patients with PCD disease-causing variants share common features, including chronic rhinitis and sinusitis, chronic cough, bronchiectasis, and low nNO level.

The current data suggest that low nNO in PCD patients is, in part, caused by increased NO oxidation. Specifically, it is shown here that antigen stasis on the surface of cells from subjects with PCD results in increased expression of DUOX1 as a one potential source for NO oxidation as discussed above. It has previously been shown that irritant exposure decreases SOD activity in the airway (24), increasing superoxide levels. See Comhair et al., “Superoxide dismutase inactivation in pathophysiology of asthmatic airway remodeling and reactivity,” (2005) Am J Pathol 166: pp. 663-674 (2005). While airway NO concentrations are low in PCD (see FIG. 5 and above, the current evidence demonstrates that the oxidation products of NO in the PCD airway are high.

In addition to contributing to low NO concentrations in the PCD airway, oxidative stress can contribute to airway injury. Superoxide reacts rapidly with NO to form ONOO⁻/ONOOH (pKa ˜6.5), and ONOOH reacts with protein tyrosines to cause protein injury through tyrosine nitration. See Marozkina et al., “Nitrogen chemistry and lung physiology,” (2015) Annu Rev Physiol 77: pp. 431-452; Haddad et al., “Quantitation of nitrotyrosine levels in lung sections of patients and animals with acute lung injury,” (1994) J Clin Invest 94: pp. 2407-2413. It is shown herein that this occurs in the PCD cells to a greater extent than HC cells (see FIG. 3). Depending on conditions in the airways, additional oxidation products, such as NO₂, and HNO₂⁻/NO₂⁻ can also be cytotoxic. Moreover, DUOX1 increases the generation of H₂O₂. It is demonstrated herein that breath H₂O₂ concentrations are increased in PCD. H₂O₂ injures the airway in part through its reaction with chloride, catalyzed by myeloperoxidase (MPO), to form HOCl (hypochlorous acid). See Kettle et al., “Myeloperoxidase and protein oxidation in the airways of young children with cystic fibrosis,” (2004) Am J Respir Crit Care Med 170: pp. 1317-1323. This is modeled in FIG. 6.

Since, H₂O₂ can also injure the airway through reaction with bromide, catalyzed by eosinophil peroxidase, to form hypobromous acid (HOBr) (see Wedes et al., “Urinary bromotyrosine measures asthma control and predicts asthma exacerbations in children,” (2011) J Pediatr 159: pp. 248-255). Thus, these data indicate that the increase in acidification of the airway in compromised lung function such as PCD, can be treated by modulating airway pH. Specifically, the inventors propose that airway pH can by modulating by increasing airway pH. More specifically, the inventors propose that compromised lung function can be treated by inhalation of an agent that increases airway pH, for example with inhalation of NaHCO3 (8.4%) or through administration of noninvasive ventilation.

FIG. 6 is a model of nitrosative and oxidative stress in PCD. It shows that in the healthy airway (right), ciliary function is normal. Antigens and irritants are rapidly cleared, and NO enters the gas phase normally to be exhaled. As disclosed herein, in the PCD airway (left), antigens and irritants are not as well cleared. This defect leads to oxidative stress, marked by increased DUOX1 expression and decreased superoxide dismutase [SOD] activity. H₂O₂ injures the airway in part through its reaction with chloride, catalyzed by myeloperoxidase (MPO), to form HOCl (hypochlorous acid). H₂O₂ is exhaled at higher concentrations. NO is oxidized rather than exhaled, forming cytotoxic intermediates.

There are additional mechanisms by which NO may be oxidized in the PCD epithelium. For example, end-expired oxygen levels are higher in PCD epithelia than normal subjects, or in subjects with cystic fibrosis (CF). See Mendelsohn et al., “A novel, noninvasive assay shows that distal airway oxygen tension is low in cystic fibrosis, but not in primary ciliary dyskinesia,” (2019) Pediatr Pulmonol 54: pp. 27-32. Note that the airways of CF subjects have increased oxygen consumption compared to disease-free epithelium. See Worlitzsch et al., “Effects of reduced mucus oxygen concentration in airway Pseudomonas infections of cystic fibrosis patients,” (2002) J Clin Invest 109: pp. 317-325. It is possible that the PCD epithelium, by contrast, may have decreased metabolic needs and decreased oxygen consumption. Further, the inventors have previously shown that the products of eNOS activation—independently of decreased eNOS expression—are regulated in part by the redox state of somatic cell Hb in the airway epithelium: heme iron oxidation to Fe (III) decreases NO concentrations but increases NO₃⁻ when eNOS is activated in the epithelium See Marozkina et al., “Somatic cell hemoglobin modulates nitrogen oxide metabolism in the human airway epithelium,” (2021) Sci Rep 11, 15498 (2021).

There are other potential reasons for decreased nNO in PCD, but none are compelling. Denitrifying organisms colonizing the airway lead to a modest (low ppb) decrease airway NO in CF and could contribute to decreased nNO in PCD, with the caveats that i) these organisms are generally less common colonizers in PCD than in CF, and ii) airway NO levels are overall somewhat higher in CF than in PCD. See Gaston et al., “Nitrogen redox balance in the cystic fibrosis airway: effects of antipseudomonal therapy,” (2002) Am J Respir Crit Care Med 165: pp. 387-390; Wijers et al., “Bacterial infections in patients with primary ciliary dyskinesia: Comparison with cystic fibrosis,” (2017) Chron Respir Dis 14: pp. 392-406. Airway pH can affect airway NO levels, but PCD patients (unlike CF patients) have no intrinsic reason to have low airway epithelial surface pH. Arginine, citrulline and ADMA metabolism are not known to be disordered in PCD patients; nor is S-nitrosothiol metabolism: these factors are unlikely to cause uniformly low nNO production rate values in PCD relative to HC or to other conditions. See Marozkina et al., “Nitrogen chemistry and lung physiology,” (2015) Annu Rev Physiol 77: pp. 431-452.

The current disclosure underscores the importance of airway clearance, exercise and cough in moving antigens out of the PCD airway. Antigen stasis and oxidative/nitrosative stress appear likely to injure the airway epithelium. The invention provides a method to determine the efficacy of airway clearance over time by fractional nitric oxide concentration and hydrogen peroxide in the exhaled breath of the patient before the initiation of treatment and at various timepoints following the initiation of treatment. Thus, an increase in fractional nitric oxide concentration and a decrease in hydrogen peroxide in the exhaled breath of the patient over time indicate positive efficacy of treatments such as antioxidant therapy which can be used in patients with PCD and other patients with compromised lung function.

As will be appreciated from the descriptions herein, a wide variety of aspects and embodiments are contemplated by the present disclosure, examples of which include, without limitation, the aspects and embodiments listed below:

The current invention provides novel approaches to the treatment of compromised lung function and methods for measuring or assessing the effectiveness of such treatments.

More specifically, the current invention provides:

Methods of assessing the efficacy of therapies used in the treatment of a patient with compromised lung function comprising measuring fractional nitric oxide concentration and hydrogen peroxide in the exhaled breath of the patient before the initiation of treatment and at various timepoints following the initiation of treatment.

Methods of assessing the efficacy of therapies used in the treatment of a patient with compromised lung function comprising measuring fractional nitric oxide concentration and hydrogen peroxide in the exhaled breath of the patient before the initiation of treatment and at various timepoints following the initiation of treatment where the therapy or therapies comprise antioxidant therapy or the administration of one or more antioxidants.

Methods of assessing the efficacy of therapies used in the treatment of a patient with compromised lung function where an increase in fractional nitric oxide concentration and a decrease in hydrogen peroxide in the exhaled breath of the patient over time indicate positive efficacy of the antioxidant therapy.

Methods of assessing the efficacy of therapies used in the treatment of a patient with compromised lung function where the therapy is the administration of one or more antioxidants administered to a patient with compromised lung function, and where the one or more of the antioxidants are selected from the group consisting of N-acetylcysteine, Nacystelyn, N-isobutyrlcysteine, carbocisteine, procysteine, erdosteine, thioredoxin, 15d-PGJ2, CDDO-imidazolide, sulforaphane, chalcones, superoxide dismutase, ebselen, molecular hydrogen, celastrol, 2-thioxanthine, glutathione, vitamin A (retinol) β-carotenes, vitamin C (ascorbic acid), vitamin D (cholecalciferol), vitamin E (gamma gamma and/or alpha tocopherol), lipo-glutathione (glutathione) and coenzyme Q (ubiquinone).

Methods of assessing the efficacy of therapies used in the treatment of a patient with compromised lung function where the therapy is the administration of one or more antioxidants administered to a patient with compromised lung function, and where the one or more of the antioxidants are administered by one or more of routes of administration wherein the route of administration is selected from the group consisting of oral, inhalation, intravenous, subcutaneous, sublingual, and transdermal.

Methods of assessing the efficacy of therapies used in the treatment of a patient with compromised lung function where “compromised lung function” is considered to be is considered to be primary ciliary dyskinesia. In any embodiment the primary ciliary dyskinesia can be exacerbated by one or more of asthma, exposure to cigarette smoke, exposure to atmospheric pollutants, chronic obstructive pulmonary disease, bronchitis, cystic fibrosis, extended post-viral bronchial hyperresponsiveness syndrome, rhinosinusitis, reactive airways dysfunction syndrome, and persistent allergen exposure.

Methods of treating compromised lung function in a patient in need thereof wherein the compromised lung function is caused by primary ciliary dyskinesia, and where the methods comprise the administration of a therapy that modulates airway pH.

Methods of treating compromised lung function in a patient in need thereof wherein the compromised lung function is caused by primary ciliary dyskinesia, and where the methods comprise administration of a therapy that modulates airway pH where the therapy that modulates airway pH increases airway pH.

Methods of treating compromised lung function in a patient in need thereof wherein the compromised lung function is caused by primary ciliary dyskinesia, and where the methods comprise administration of a therapy that modulates airway pH where the therapy that modulates airway pH is nebulized NaHCO₃.

Methods of treating compromised lung function in a patient in need thereof wherein the compromised lung function is caused by primary ciliary dyskinesia, and where the methods comprise administration of a therapy that modulates airway pH where the therapy that modulates airway pH is administration of noninvasive ventilation.

Methods of treating compromised lung function in a patient in need thereof wherein the compromised lung function is caused by primary ciliary dyskinesia, and where the methods comprise administration of a therapy that modulates airway pH further comprising the administration of one or more of an antioxidant.

Methods of assessing the efficacy of the treatment of compromised lung function in a patient, thereof wherein the compromised lung function is caused by primary ciliary dyskinesia and wherein the treatment comprises administration of an agent that modulates airway pH where the method comprises measuring fractional nitric oxide concentration and hydrogen peroxide in the exhaled breath of the patient before the initiation of treatment and at various timepoints following the initiation of treatment.

Methods of assessing the efficacy of therapies used in the treatment of a patient with compromised lung function thereof wherein the compromised lung function is caused by primary ciliary dyskinesia where an increase in fractional nitric oxide concentration and a decrease in hydrogen peroxide in the exhaled breath of the patient over time indicate positive efficacy of treatment that modulates airway pH.

Methods of assessing the efficacy of the treatment of compromised lung function in a patient, wherein the compromised lung function is caused by primary ciliary dyskinesia and where the treatment comprises administration of a therapy that modulates airway pH where the a therapy that modulates airway pH increases airway pH.

Methods of assessing the efficacy of the treatment of compromised lung function in a patient wherein the compromised lung function is caused by primary ciliary dyskinesia and where the treatment comprises administration of a therapy that modulates airway pH where the therapy that modulates airway pH increases airway pH and where that therapy is nebulized NaHCO₃.

Methods of assessing the efficacy of the treatment of compromised lung function in a patient wherein the compromised lung function is caused by primary ciliary dyskinesia and where the treatment comprises administration of a therapy that modulates airway pH where the therapy that modulates airway pH increases airway pH and where that therapy the administration of noninvasive ventilation.

Methods of assessing the efficacy of the treatment of compromised lung function in a patient wherein the compromised lung function is caused by primary ciliary dyskinesia and where the treatment comprises administration of a therapy that modulates airway pH or where the treatment comprises administration of administration of one or more antioxidants and where the method further comprises the administration of one or more additional therapeutic agents.

Methods of assessing the efficacy of the treatment of compromised lung function in a patient wherein the compromised lung function is caused by primary ciliary dyskinesia and where the treatment comprises administration of a therapy that mechanically augments mucociliary clearance, moving antigens and irritants into and out of the airways.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of assessing the efficacy of antioxidant therapy in a patient with primary ciliary dyskinesia comprising measuring fractional nitric oxide concentration and hydrogen peroxide in the exhaled breath of the patient before the initiation of treatment and at various timepoints following the initiation of treatment.

2. The method of claim 1, wherein an increase in fractional nitric oxide concentration and a decrease in hydrogen peroxide in the exhaled breath of the patient over time indicate positive efficacy of the antioxidant therapy.

3. The method of claim 1, wherein the antioxidant therapy in a patient with primary ciliary dyskinesia comprises the administration of one or more of the agents selected from the group consisting of N-acetylcysteine, Nacystelyn, N-isobutyrlcysteine, carbocisteine, procysteine, erdosteine, thioredoxin, 15d-PGJ2, CDDO-imidazolide, sulforaphane, chalcones, superoxide dismutase, ebselen, molecular hydrogen, celastrol, 2-thioxanthine, glutathione, vitamin A (retinol) β-carotenes, vitamin C (ascorbic acid), vitamin D (cholecalciferol), vitamin E (gamma and/or alpha tocopherol), lipo-glutathione (glutathione) and coenzyme Q (ubiquinone).

4. The method of claim 3, wherein the antioxidant therapy is administered by one or more of routes of administration wherein the route of administration is selected from the group consisting of oral, inhalation, intravenous, subcutaneous, sublingual, and transdermal.

5. The method of claim 1, wherein the primary ciliary dyskinesia is exacerbated by one or more of asthma, exposure to cigarette smoke, exposure to atmospheric pollutants, chronic obstructive pulmonary disease, bronchitis, cystic fibrosis, extended post-viral bronchial hyperresponsiveness syndrome, rhinosinusitis, reactive airways dysfunction syndrome, and persistent allergen exposure.

6. A method of treating primary ciliary dyskinesia in a patient in need thereof comprising a therapy that modulates airway pH.

7. The method of claim 6, wherein the therapy that modulates airway pH increases airway pH.

8. The method of claim 6, wherein the therapy that modulates airway pH is nebulized NaHCO₃.

9. The method of claim 6, wherein the therapy that modulates airway pH is noninvasive ventilation.

10. The method of claim 3, further comprising a therapy that modulates airway pH.

11. The method of claim 6, wherein the primary ciliary dyskinesia is exacerbated by one or more of one or more of asthma, exposure to cigarette smoke, exposure to atmospheric pollutants, chronic obstructive pulmonary disease, bronchitis, cystic fibrosis, extended post-viral bronchial hyperresponsiveness syndrome, rhinosinusitis, reactive airways dysfunction syndrome, and persistent allergen exposure.

12. A method of assessing the efficacy of treatment of primary ciliary dyskinesia in a patient, wherein the treatment comprises administration of a therapy that modulates airway pH, comprising measuring fractional nitric oxide concentration and hydrogen peroxide in the exhaled breath of the patient before the initiation of treatment and at various timepoints following the initiation of treatment.

13. The method of claim 12, wherein an increase in fractional nitric oxide concentration and a decrease in hydrogen peroxide in the exhaled breath of the patient over time indicate positive efficacy of the treatment with a therapy that modulates airway pH.

14. The method of claim 12, wherein the therapy that modulates airway pH increases airway pH.

15. The method of claim 14, wherein the therapy that modulates airway pH is nebulized NaHCO₃.

16. The method of claim 14, wherein the therapy that modulates airway pH is noninvasive ventilation.

17. The method of claim 12, further comprising the administration of one or more of an antioxidant.

18. The method of claim 17, where in the antioxidant is selected from the group consisting of N-acetylcysteine, Nacystelyn, N-isobutyrlcysteine, carbocisteine, procysteine, erdosteine, thioredoxin, 15d-PGJ2, CDDO-imidazolide, sulforaphane, chalcones, superoxide dismutase, ebselen, molecular hydrogen, celastrol, 2-thioxanthine, glutathione, vitamin A (retinol) β-carotenes, vitamin C (ascorbic acid), vitamin D (cholecalciferol), vitamin E (gamma tocopherol), lipo-glutathione (glutathione) and coenzyme Q (ubiquinone).

19. The method of claim 17, wherein the antioxidant therapy is administered by one or more of routes of administration wherein the route of administration is selected from the group consisting of oral, inhalation, intravenous, subcutaneous, sublingual, and transdermal.

20. The method of claim 12, wherein the primary ciliary dyskinesia is exacerbated by one or more of asthma, exposure to cigarette smoke, exposure to atmospheric pollutants, chronic obstructive pulmonary disease, bronchitis, cystic fibrosis, extended post-viral bronchial hyperresponsiveness syndrome, rhinosinusitis, reactive airways dysfunction syndrome, and persistent allergen exposure.

21. (canceled)

22. (canceled)
